(19) United States
(12) Patent Application Publication
(10) Pub. No.: US 2009/0304712 A1
(43) Pub. Date: Dec. 10, 2009

(54) Neuronal Cell Death Inhibitor and Screening Method

(75) Inventors: Hideyuki Takeuchi, Nagoya-shi (JP); Akio Suzumura, Nagoya-shi (JP)

Correspondence Address: OLIFF & BERRIDGE, PLC, P.O. BOX 320850, ALEXANDRIA, VA, 22320-4850, US

(73) Assignee: NATIONAL UNIVERSITY CORPORATION NAGOYA UNIVERSITY, AICHI (JP)

(21) Appl. No.: 12/223,472

(22) PCT Filed: Feb. 1, 2007
(86) PCT No.: PCT/JP2007/000050

§ 371 Date: May 7, 2009

(30) Foreign Application Priority Data
Feb. 2, 2006 (JP) 2006-026144

(51) Int. Cl. A61K 39/395; G01N 33/553; A61P 25/00

(52) U.S. Cl. 424/172.1; 435/7.21

(57) ABSTRACT

A neuronal cell death inhibitor comprising a compound having an inhibitory activity on the production and/or release of glutamic acid in a microglia; by inhibiting the production and/or release in a microglia, neurite bead-like degeneration or neuronal cell death can be inhibited.

### TECHNICAL FIELD

The present invention relates to neuronal cell death inhibitors that restrain or avoid neuronal cell death by glutamate.

### BACKGROUND

A variety of studies have been tried to develop the prevention and treatment of neurodegenerative diseases such as Alzheimer's disease, Parkinson's disease, amyotrophic lateral sclerosis, spinocerebellar degeneration, multiple sclerosis and the like. Microglial activation contributes to the neurotoxicity observed in those neurodegenerative diseases. Excito-neurotoxicity by glutamate released from activated microglia is considered as a major cause of these neurodegenerative diseases (Block et al., Prog. Neurobiol. 76, 77-98 (2005)). Thus, blockade of glutamate receptors is considered as a promising therapy for neurodegenerative diseases (Parsons et al., Neuropharmacology. 38, 735-767 (1999)). Inhibition of microglial activation is another therapeutic candidate for neurodegenerative diseases (Demercq et al., Trends. Pharmacol. Sci. 25, 609-612 (2004)).

However, glutamate receptor inhibitors reportedly induce serious adverse effects in a dose dependent manner, because glutamate receptor inhibitors not only inhibit excessive excitotoxicity but also perturb physiological glutamate signal that is essential for normal nervous conduction. In addition, inhibition of microglial activation has exhibited poor therapeutic effects because microglia also have neuroprotective effects mediated by neurotrophin release, glutamate uptake, and sequestering neurotoxic substances.

### DISCLOSURE OF THE INVENTION

The present inventors reasoned that it is difficult to obtain the intended effect using inhibitors of glutamate receptors or activated microglia due to the non-specificity thereof. In addition, they reached the idea that the specific inhibitors that inhibit only deleterious neurotoxic microglia or the production and release of excessive glutamate could prevent neuronal cell death effectively. Note that the details of the mechanism of production and release of glutamate from microglia have not been clarified so far. No drug is known, which attempts to inhibit neuronal cell death by inhibiting the generation or the release of glutamate.

One object of the present teachings is to provide drugs that inhibit or avoid neuronal cell death caused by glutamate, and a screening method for the drug. In addition, another object of the present teachings is to provide drugs that inhibit neurotoxic activated microglia or the production and release of glutamate from microglia, and a screening method for the drug.

The present inventors did not set their focus on conventional viewpoints such as the inhibition of N-methyl-D-aspartate type glutamate receptor (NMDA receptor) or the inhibition of activated microglia in its entirety, but on the mechanism of glutamate production and release from microglia, and have carried out a variety of tests regarding factors related to the amount of glutamate released in microglia. In addition, a variety of tests were carried out simultaneously on the relationship between glutamate release and neuritic beading degeneration and neuronal cell death. As the results of those examinations, it was discovered that an inhibition of microglial production and/or release, i.e. any among an inhibition of glutaminase, an inhibition of gap junction hemichannels in microglia and an inhibition of microglia activation by tumor necrosis factor (TNF-α) or the like, affords suppression of glutamate production or decrease in the amount release thereof; and, it was further discovered that such inhibitions of microglial production and/or release efficiently inhibit neuritic beading degeneration and neuronal cell death. The present invention was completed based on the aforementioned epochal discoveries. That is to say, according to the present teachings, the following teachings are provided.

According to the present teachings, neuronal cell death inhibitor containing a compound having inhibitory activity that inhibits the production and/or release of glutamate in microglia is provided.

A preferred mode is that the above compound in this cell death inhibitor has an activity of inhibiting activated production and/or release of glutamate from microglia. The compound may be a glutaminase inhibitor, e.g. it may be (S)-2-amino-6-diazo-5-oxocaproic acid or a salt thereof.

Furthermore, the compound may be a gap junction inhibitor, e.g. it may be carbenoxolone disodium.

Furthermore, the compound may be a tumor necrosis factor inhibitor or tumor necrosis factor receptor inhibitor. Specifically, it may be a TNF-α inhibitor or a TNFR inhibitor; for the tumor necrosis factor inhibitor, anti-TNF-α antibody or soluble TNF-α receptor may be cited, and, for tumor necrosis factor receptor inhibitor, anti-TNFR1 receptor antibody or TNF-α antagonist may be cited.

Such compound preferably has an inhibitory activity that inhibits glutamate production and/or release from activated microglia to be within a range that maintains the produced amount of glutamate to approximately level with the amount of glutamate produced when microglia is not activated.

The cell death inhibitor of the present invention can be neuronal cell death inhibitor for glutamate-induced excitotoxic neurodegeneration. In addition, a preferred mode of the cell death inhibitor of the present invention is an agent for preventing and treating a nervous system disease, and as of the nervous system disease, it may be selected from ischemic disorder, neuroinflammatory disease and neurodegenerative disease. As the ischemic disorder, cerebral stroke, brain hemorrhage, cerebral infarction and cerebrovascular dementia may be cited; as the neuroinflammatory disease, acute disseminated encephalomyelitis, sequelae of encephalitis, bacterial meningitis, tuberculous meningitis, fungal meningitis, viral meningitis and post-vaccinal meningitis may be cited. Moreover, as the neurodegenerative disease, it may be selected from Alzheimer's disease, Parkinson's disease, amyotrophic lateral sclerosis, spinocerebellar degeneration, multiple system atrophy and multiple sclerosis.

According to the present invention, a composition for the prevention and treatment of diseases related to neuronal cell death, of which contains a cell death inhibitor described as in any of the above and a pharmacologically acceptable formulation constituent is provided.

According to the present teachings, a screening method for a neuronal cell death inhibitor that evaluates effects of the neuronal cell death inhibitor, by taking as an indicator the action of a test compound on a pathway of glutamate production and release from microglia. The present screening method may be utilized as a screening method for a prophylactic and therapeutic agent against nervous system diseases.

In this screening method, the above action is preferably a glutamate production or release inhibition action of the test compound with respect to such production and release by activated microglia. Further, the action may be at least one of a glutaminase inhibition action of the test compound, a gap junction inhibition action of the test compound on microglia, and a microglia activation inhibition action of the test compound on microglia. Although having any of the aforesaid inhibitory actions is sufficient, the glutaminase inhibition action or the gap junction inhibition action is more preferable. The present screening method may be provided with a step of supplying a test compound to an activated microglia in the presence of glutamine; a step of acquiring the indicator regarding microglia; and a step of determining that the test compound has neuronal cell death inhibitory activity in a case where the indicator, in comparison to its state in which the test compound is not supplied, has significantly changed to an extent that allows the neuronal cell death inhibitory activity to be affirmed.

In addition, this screening method may further evaluate the effect of a neuronal cell death inhibitor by utilizing the action of a test compound on one species or two or more species selected from the following (a) to (d) as the indicator:

(a) neuritic beading degeneration;
(b) cell death;
(c) intracellular ATP concentration; and
(d) mitochondrial damage
in neurons under the presence of activated microglia, or activated microglia conditioned medium thereof and the test compound.

### BRIEF DESCRIPTION OF THE DRAWINGS

FIG. 1 shows an overview of the pathways of glutamate production and release, and inhibition method thereof;

FIG. 2 shows a graph showing the percentage of neurons with neuritic beading degeneration among total neurons that have been cultured with conditioned medium of microglia activated by various cytokines and the like; with the proviso that white bars indicate groups in which neurons were directly stimulated by reagents (direct stimulation groups), and black bars indicate groups in which neurons were cultured with reagent-treated microglia conditioned medium (indirect stimulation groups) (*, p<0.05 versus control; **, p<0.01 versus control; †, p<0.01 versus neurons incubated with lipopolysaccharide (LPS)- or TNF-α-stimulated microglia conditioned medium; these data were analyzed by one way analysis of variance and Tukey-Kramer post-hoc test; each bar is represented by the mean value and standard deviation of six independent separate data; likewise in FIG. 3 below);

FIG. 3 shows the percentage of dead neurons among total neurons. White bars indicate direct stimulation groups, and black bars indicate indirect stimulation groups;

FIG. 4 shows phase contrast microscopic images: A shows non-stimulated microglia, B shows LPS-stimulated microglia, C shows TNF-α-stimulated microglia, D shows neurons incubated with non-stimulated microglia conditioned medium, E shows neurons incubated with LPS-treated microglia conditioned medium, and F shows neurons incubated with TNF-α-treated microglia conditioned medium (scale bar is 10 μm);

FIG. 5 shows glutamate concentration in neuron culture medium; with the proviso that white bars indicate groups in which neurons were directly stimulated by reagents (direct stimulation groups), and black bars indicate groups in which neurons were cultured with reagent-treated microglia conditioned medium (indirect stimulation groups) (*, p<0.05 with respect to versus control;**, p<0.01 with respect to versus control; †, p<0.01 with respect to neurons cultured in lipopolysaccharide (LPS)- or TNF-α-stimulated microglia culture supernatant; these data were analyzed by one way analysis of variance and Tukey-Kramer post-hoc test; each bar is represented by the mean value and standard deviation of six independent separate data; likewise in FIGS. 6 and 7 below);

FIG. 6 shows intracellular ATP concentration in neurons. White bars indicate direct stimulation groups, and black bars indicate indirect stimulation groups;

FIG. 7 shows MTS assay for neurons. White bars indicate direct stimulation groups, and black bars indicate indirect stimulation groups;

FIG. 8 shows glutamate concentration in a neuron culture medium, which has been cultured with activated microglia conditioned medium and various neutralizing antibodies (a-TNF, anti-TNF-α neutralizing antibody; a-R1, anti-TNFR1 neutralizing antibody; a-R2, anti-TNFR2 neutralizing antibody; TNF1, 1 ng/ml TNF-α; TNF10, 10 ng/ml TNF-α; TNI100, 100 ng/ml TNF-α. *, p<0.05 versus control; **, p<0.01 versus control; †, p<0.05 versus neurons incubated with LPS- or TNF-α-treated microglia conditioned medium; these data were analyzed by one way analysis of variance and Tukey-Kramer post-hoc test; each bar is represented by the mean value and standard deviation of six independent separate data; likewise in FIG. 9 and FIG. 10 below);

FIG. 9 shows the percentage of neurons with neuritic beading degeneration among total neurons that have been cultured with activated microglia conditioned medium and various neutralizing antibodies;

FIG. 10 shows the percentage of dead neurons among total neurons that have been cultured with activated microglia conditioned medium and various neutralizing antibodies;

FIG. 11 shows glutamate concentration in neuron culture medium, which has been incubated with activated microglia conditioned medium and various drugs (*, p<0.05 versus control; †, p<0.05 versus neurons incubated with LPS- or TNF-α-stimulated microglia conditioned medium; these data were analyzed by one way analysis of variance and Tukey-Kramer post hoc test; each bar is represented by the mean value and standard deviation of six independent separate data; likewise in FIG. 12 and FIG. 13 below);

FIG. 12 shows the percentage of neurons with neuritic beading degeneration among total neurons that have been cultured with activated microglia conditioned medium and various drugs;

FIG. 13 shows the percentage of dead neurons among total neurons that have been cultured with activated microglia conditioned medium and various drugs;

FIG. 14 shows flow cytometric data of microglial cell surface expression of connexin-32 (C×32), which is a major constitutive factor of gap junction;

FIG. 15 shows the effects of carbenoxolone (CBX), which is a gap junction inhibitor, and 6-diazo-5-oxo-L-norleucine (DON), which is a glutaminase inhibitor, on ischemia-induced delayed neuronal cell death. A to H show micrographic images of the gerbil hippocampal CA1 regions (scale bar: 100 μm): A shows a normal group, B shows an ischemia group administered with PBS, C shows an ischemia group administered with 0.2 mg/kg body weight of CBX (CBX1/100), D shows an ischemia group administered with 2 mg/kg body weight of CBX (CBX1/10), E shows an ischemia group administered with 20 mg/kg body weight of CBX (CBX1), F shows an ischemia group administered with 0.016 mg/kg body weight of DON (DON1/100), G shows an ischemia group administered with 0.16 mg/kg body weight of DON (DON1/10), and H shows an ischemia group administered with 1.6 mg/kg body weight of DON (DON1), respectively;

FIG. 16 shows the number of surviving neurons per 100 μm of gerbil hippocampal CA1 region in A to H of FIG. 15. *, p<0.001 versus PBS-administered group; †, p<0.001. These data were analyzed by one way analysis of variance and Tukey-Kramer post-hoc test. Each bar is represented by the mean value and standard deviation of three independent separate data;

FIG. 17 shows the effects of carbenoxolone (CBX) and 6-diazo-5-oxo-L-norleucine (DON) on experimental autoimmune encephalomyelitis (EAE) mice. A shows the EAE clinical score for the CBX-administered group: PBS shows an EAE group administered with PBS, CBX1/100 shows an EAE group administered with 0.2 mg/kg body weight of CBX, CBX1/10 shows an EAE group administered with 2 mg/kg body weight of CBX, CBX1 shows an EAE group administered with 20 mg/kg body weight of CBX. B shows the EAE clinical score for the DON-administered group: PBS shows an EAE group administered with PBS, DON1/100 shows an EAE group administered with 0.016 mg/kg body weight of DON, DON1/10 shows an EAE group administered with 0.16 mg/kg body weight of DON, DON1 shows an EAE group administered with 1.6 mg/kg body weight of DON;

FIG. 18 shows the onset day of each administered group obtained from the EAE clinical score shown in A and B of FIG. 17;

FIG. 19 shows the number of severe sick days (clinical score is four or greater) of each administered group obtained from the EAE clinical score shown in A and B of FIG. 17; and

FIG. 20 shows the peak clinical score of each administered group obtained from the EAE clinical score shown in A and B of FIG. 17. *, p<0.05 versus PBS-administered group. These data were analyzed by one way analysis of variance and Tukey-Kramer post-hoc test. Each bar is represented by the mean value and standard deviation of five independent separate data.

### BEST MODE FOR CARRYING OUT THE INVENTION

The present invention is related to a neuronal cell death inhibitor containing a compound having inhibitory activity of inhibiting the production and/or release of glutamate from microglia. The present inventors obtained the observations that the augmentation of the number of dead neurons, or the like, caused by TNF-α-stimulated microglia conditioned medium is relates to augmentation of the amount of glutamate released from similarly activated microglia and augmentation of mitochondrial disturbances in neurons. The present inventors also obtained the observations that the amount of glutamate released from microglia, the number of dead neurons, and the like, are diminished by the TNF-neutralizing antibody or the TNF receptor-neutralizing antibody; and moreover, that the amount of glutamate released from activated microglia, the number of dead neurons, and the like, are decreased by glutamine deprivation in the culture medium, a glutaminase inhibitor and a gap junction inhibitor. Moreover, the present inventors obtained the observation that the migration property and the expression of gap junctions are markedly enhanced in microglia activated by TNF-α or the like. Therefore, gap junctions of activated microglia are more openly exposed to the extracellular space since enhancement of migration property is associated with decrease in intercellular adhesions.

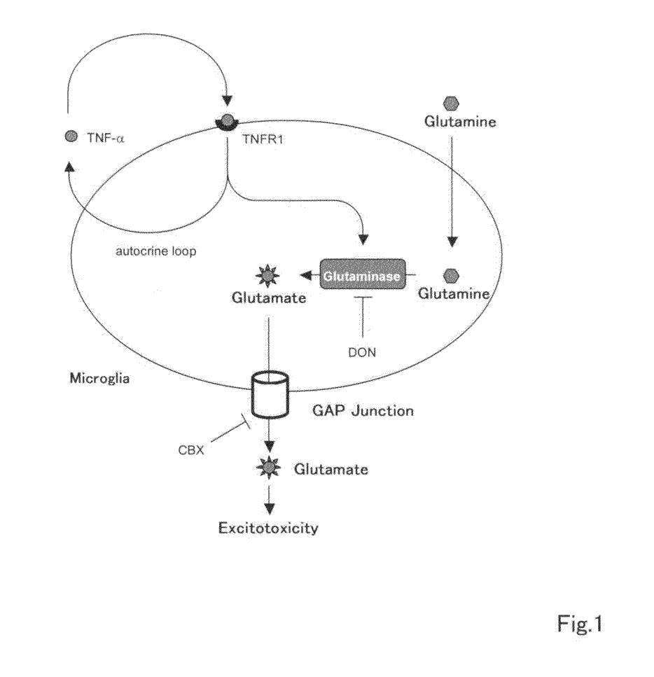

According to such observations, the present inventors proposed a scheme of glutamate production and release by activated microglia, as shown in FIG. 1, and an inhibition method for this scheme. That is to say, microglial glutaminase activated by TNF-α or LPS produces glutamate from extracellular glutamine as a substrate, and this produced glutamate is released outside of microglia through gap junctions. The glutamate produced and released in this pathway binds to the NMDA receptor of neurons, inducing neuronal cell death via intracellular ATP starvation by mitochondrial respiratory inhibition. In addition, TNF-α also has the activity of promoting the release of TNF-α from microglia in an autocrine manner.

According to the present inventors, excessive glutamate production and release from activated microglia can be selectively inhibited by blocking such glutamate production and release pathway. According to such selective inhibition, neuronal cell death can be rescued without perturbing normal glutamate activities, as basal production of glutamate is not inhibited.

Hereinafter, neuronal cell death inhibitor, application thereof and screening method for cell death inhibitor will be described, which are embodiments of the present teachings.

### (Neuronal Cell Death Inhibitor)

The cell death inhibitor of the present invention contains a compound having the inhibitory activity of inhibiting glutamate production and/or release from microglia (hereinafter, simply referred to as glutamate release inhibitor).

In the present invention, “neuronal cell death” includes both necrosis and apoptosis. Necrosis means death occurring to a batch of cells in a pathologically state such as ischemia, and dissolution and autolysis of cells may be cited due to a variety of external factors. Meanwhile, apoptosis means the dying state of a cell, which activates a mechanism to kill itself spontaneously due to a variety of causes, such as turning over cells in a healthy tissue of an animal and during elimination of cells that are unnecessary in the development stage of a variety of organs.

As the glutamate release inhibitor in the present invention, those capable of inhibiting the production and/or release of glutamate in activated microglia is desirable, and as compounds in such mode, a glutaminase inhibitor, a gap junction inhibitor and a microglia activation inhibitor may at the least be cited. According to these glutamate release inhibitors, the glutamate production and/or release in activated microglia can be inhibited so that the amount of glutamate produced is maintained within a range approximately equal to the amount under a state in which microglia is not activated. The cell death inhibitor of the present invention can contain one species of such various glutamate release inhibitors, or two or more species in combination.

### (1) Glutaminase Inhibitor

A glutaminase inhibitor suffices to be a compound that inhibits glutaminase, which is an enzyme that generates glutamate from glutamine. The inhibition mode is not limited in particular. As glutaminase inhibitors, well-known glutaminase inhibitors can be used, with no particular limitation. For instance, 6-diazo-5-oxo-L-norleucine ((S)-2-amino-6-diazo-5-oxocaproic acid or a salt thereof (DON)) and the species of imidazole derivatives described in Published Japanese Patent Application Laid-open No. H7-188181 may be cited. A glutaminase inhibitor can inhibit production of excessive glutamate in activated microglia, therefore is desirable as the glutamate release inhibitor of the present invention.

### (2) Gap Junction Inhibitor

A gap junction inhibitor suffices to be a compound that inhibits intercellular communication such as movement and exchange of low molecular weight compounds, or the like, via the pore of a channel of a gap junction. As gap junction inhibitors, well-known gap junction inhibitors can be used. For instance, various fatty acid primary amide compounds, e.g. oleamide or arachidonamide, which is a species of oleamide agonist (for instance, Published Japanese translation of PCT International Publication Laid-open No. 2001-523695), carbenoxolone or a salt such as carbenoxolone disodium, 18α-glycyrrhizin acid or a salt thereof, 12-O-tetradecanoylphorbol-13-acetate (TPA), octanol or lindane may be cited. In addition, agonists of connexin 40 and 43 such as ⁴³GAP27 peptide (SRPTEKTIFII) and ⁴⁰GAP27 peptide (SRPTEKNVFIV), a species of cAMP and/or cAMP phosphodiesterase inhibitor described in Published Japanese translation of PCT International Publication Laid-open No. 2005-509621, a species of glycosaminoglycan described in Published Japanese Patent Application Laid-open No. 2004-217594, and the like, can also be cited. A gap junction inhibitor can inhibit glutamate release during production of excessive glutamate in activated microglia, and therefore is desirable as the glutamate release inhibitor of the present invention.

### (3) Microglia Activation Inhibitor

As microglia activation inhibitor, a compound that inhibits the stimulation transmission by a cytokine, which activates glutamate production and release by microglia, is desirable. For instance, an inhibitor of TNF-α or a receptor inhibitor that inhibits binding of TNF-α in the receptor thereof may be cited. As such inhibitors, compounds that have TNF-α or TNF-α Receptor type 1 (TNFR1) as the target and inhibit the binding between TNF-α and the receptor may be cited. Specifically, various well-known compounds, e.g. anti-TNF-α antibody, soluble TNFR1 receptor, anti-TNFR1 antibody, and TNF-α antagonist e.g. WP9QY may be cited. Note that, not only can these inhibitors inhibit microglia activation by TNF-α, but they can also inhibit activation by LPS.

In addition, LPS inhibitors that are competitive inhibitors (E5531 and E5564) of Toll-Like-Receptor4 (TLR4), which is an LPS receptor, or TLR4 neutralizing antibodies, can also be used.

Such various glutamate release inhibitors can be in various salt forms, as necessary, depending on the forms of the acidic groups and basic groups of the compound thereof. Such salt forms can be constituted using hydrochloric acid or bases commonly used in the field of medicine or the like.

The cell death inhibitor of the present invention contains a glutamate production and release inhibitor, such that it is preferably used as a cell death inhibitor for excito-neurotoxiciy caused by glutamate. In addition, it is preferably used as an agent for the prevention and treatment of nervous system diseases of human and non-human animals, such as livestock and pets, related to neuronal cell death caused by such excito-neurotoxiciy. As nervous system diseases, for instance, ischemic disorders, neuroinflammatory diseases, neurodegenerative diseases, and the like, may be cited.

As ischemic disorders, for instance, cerebral stroke, brain hemorrhage, cerebral infarction and cerebrovascular dementia may be cited. As neuroinflammatory diseases, for instance, central nervous system inflammatory nervous diseases, such as, sequelae of encephalitis, acute disseminated encephalomyelitis, bacterial meningitis, tuberculous meningitis, fungal meningitis, viral meningitis and post-vaccinal meningitis may be cited. As neurodegenerative diseases, for instance, Alzheimer's disease, head injury, cerebral palsy, Huntington's disease, Pick's disease, Down's syndrome, Parkinson's disease, AIDS encephalopathy, multiple system atrophy, multiple sclerosis, amyotrophic lateral sclerosis, spinocerebellar degeneration and the like, may be cited.

When using the cell death inhibitor of the present invention as an agent for the prevention and treatment of such nervous system diseases as above of human and non-human animals related to neuronal cell death, it can be per se or mixed with a suitable pharmacologically acceptable formulation constituent, such as excipient, diluent or the like, to be constituted as a composition (formulation) such as tablet, encapsulated formulation, granule, powdered drug or syrup agent. That is to say, a composition for the prevention and treatment of a nervous system disease having the neuronal death inhibitor of the present invention as an active ingredient is provided. Depending on the formulation to be obtained, the present composition can contain a pharmacologically acceptable formulation constituent, in addition to the active ingredient. The prevention and treatment composition of the present invention can be administered perorally or parenterally.

These formulations are prepared by widely known methods, using additives, such as, excipients (for instance, organic series excipients, such as, sugar derivatives, such as, lactose, sucrose, glucose, mannitol and sorbitol; starch derivatives, such as corn starch, potato starch, a starch and dextrin; cellulose derivatives such as crystalline cellulose; gum arabic; dextran; and pullulan; and inorganic series excipients such as, silicate derivatives such as light anhydrous silicic acid, synthetic aluminum silicate, calcium silicate and magnesium aluminometasilicate; phosphoric acid salts such as calcium hydrogen phosphate; carbonates such as calcium carbonate; and sulfates such as calcium sulfate can be cited), lubricants (for instance, stearic acid and metal salts of stearic acid such as calcium stearate and magnesium stearate; talc; colloidal silica; waxes such as beegum and whale wax; boric acid; adipic acid; sulfates such as sodium sulfate; glycol; fumaric acid; sodium benzoate; DL leucine; sodium salts of fatty acid; lauryl sulfates such as sodium lauryl sulfate and magnesium lauryl sulfate; silicic acids such as anhydrous silicic acid, and silicic acid hydrate; and, the above-mentioned starch derivative can be cited), binders (for instance, hydroxypropyl cellulose, hydroxypropyl methyl cellulose, polyvinylpyrrolidone, macrogol, and, similar compounds to the above excipients can be cited), disintegrants (for instance, cellulose derivatives such as, low substitution degree hydroxypropyl cellulose, carboxymethyl cellulose, calcium carboxymethyl cellulose, internally-crosslinked sodium carboxymethyl cellulose; chemically modified starch and celluloses carboxymethyl starch, sodium carboxymethyl starch and crosslinked polyvinylpyrrolidone can be cited), stabilizers (paraoxy benzoates such as methyl paraben and propyl paraben; alcohols such as chlorobutanol, benzyl alcohol and phenylethyl alcohol; benzalkonium chloride; phenols such as phenol and cresol; thimerosal; dehydro acetic acid; and, sorbic acid can be cited), flavoring agents (for instance, commonly used edulcorants, acidulants, flavors, and the like, can be cited), and diluents.

The amount of dosage depends on the symptoms, age, and the like, and is determined suitably in each case. For example, according to the symptoms, an adult can be administered daily, at once or distributed over several times, with a lower limit of 0.1 mg (preferably, 1 mg) and an upper limit of 1000 mg (preferably, 500 mg) in the case of oral administration, and a lower limit 0.01 mg (preferably, 0.1 mg) and an upper limit 500 mg (preferably, 200 mg) daily per time, in the case of intravascular administration.

### (Screening Method)

The screening method for the neuronal cell death inhibitor of the present invention is one whereby the effects of the neuronal cell death inhibitor is evaluated taking as an indicator the action of the test compound on the pathway of glutamate production and release from microglia. As has already been explained, it is known that neuronal cell death can be inhibited effectively with glutamate release inhibitor. According to the screening method of the present invention, by taking the various actions provoked by the glutamate release inhibitor as an indicator, and as a result, the effects as a cell death inhibitor can be evaluated.

As the indicator of the effects of a cell death inhibitor, the inhibition action of the test compound on the production or release of glutamate by activated microglia may be cited. Specifically, glutaminase inhibitory action of the test compound, gap junction inhibitory action of the test compound on microglia, or the inhibitory action of the test compound on microglia on microglia activation may be cited.

The glutaminase inhibitory action can be acquired, for instance, by measuring the concentration of glutamate released in the microglia conditioned medium when the test compound is supplied to activated microglia. The glutamate concentration in the microglia conditioned medium can be measured by well-known glutamate colorimetric methods and sensors. The test compound is not limited in particular, and analogs of well-known glutaminase inhibitors or the like can be used.

The gap junction inhibitory action can be acquired, for instance, by measuring the glutamate concentration in the microglia conditioned medium, or by measuring the expression level of connexin, which is a major constitutive protein of gap junction in microglia, with a flow cytometer, under the condition in which the test compound is supplied to activated microglia. The test compound is not limited in particular, and analogs of gap junction inhibitors can be used.

The inhibitory action on microglia activation can be acquired by morphological observation of the microglia (observation of the extent (degree) of microglia activation) in a state in which the test compound is supplied to activated microglia, or by measuring the glutamate concentration in the microglia conditioned medium in a state in which the test compound is supplied to activated microglia. The test compound is not limited in particular, and analogs of well-known TNF-α antagonist, anti-TNF-α antibody, soluble TNF receptor, and the like, can be used.

To carry out the screening method of the present invention, in the presence of glutamine in the culture medium, a test compound is supplied to activated microglia and any one or two or more indicators as described above are acquired in regards to the microglia. Then, when the acquired indicator has changed significantly, in comparison to its state in which the test compound is not supplied, to an extent that neuronal death inhibitory activity can be affirmed, it is determined that the test compound has a neuronal death inhibitory activity. For instance, when a significant decrease in glutamate concentration in microglia conditioned medium and a significant decrease in the extent of microglia activation by morphological observation have been obtained, the test compound can be determined to have a neuronal death inhibitory activity.

Furthermore, in the screening method of the present invention, in addition to indicators related to microglia, the action of test compound on neuron obtained through microglia can also be used as an indicator. That is to say, the effects of a neuronal cell death inhibitor can be evaluated by the action of a test compound on cell death of neurons in the presence of activated microglia conditioned medium and supplied with the test compound, or neurons co-cultured with such microglia. That is to say, when the obtained indicator has changed significantly compared to the case where the test compound has not been supplied, to a degree that neuronal cell death inhibitory activity can be affirmed, the test compound can be determined to have a neuronal cell death inhibitory activity.

As indicators of effects as a neuronal cell death inhibitor, neuronal cell damage such as neuritic beading degeneration, neuronal cell death, intracellular ATP concentration and mitochondrial damage may be cited. One species or two or more species thereof may be combined in utilization as the indicator(s).

Neuritic beading degeneration, focal bead-like swellings in dendrites and axons, is an early pathological feature of neuronal cell death triggered by activated microglia, mediated by N-methyl-D-aspartic acid type glutamate receptor (NMDA receptor) signaling (Takeuchi et al., J. Biol. Chem. 280, No. 11, 10444-10454 (2005)). Therefore, it may be an excellent indicator of neuronal cell death. Specifically, it suffices to observe neurons under a microscope or a phase contrast microscope, and determine the number of neurons with neuritic beading degeneration or the ratio among the total number of cells. For instance, when neurons with neuritic beading degeneration show a significant increase due to microglia stimulated by the test compound, the test compound can be determined to have neuronal cell death inhibitory activity.

In addition, cell death can be measured by prior art well-known methods. For instance, observation under a microscope below, further, various staining methods, for instance, the dye-exclusion method of staining dead cells using propidium iodide, or the like, ISNT (in situ nick translation) method, TUNEL (terminal deoxynucleotidyltransferase-mediated UTP end labeling) method and the like can be used suitably. For instance, when the number of dead neurons shows a significant increase due to the microglia stimulated by the test compound, the test compound can be determined to have neuronal death inhibitory activity.

The neuronal intracellular ATP concentration can be measured by well-known methods, such as, chemiluminescent method by, e.g. ApoSENSOR Cell Viability Assay Kit (manufactured by Bio Vision) or the like. In addition, for mitochondrial damages, staining method using MitoTracker Red CMXRos (manufactured by Molecular Probes) whose staining intensity is directly proportional to mitochondrial membrane potential, and tetrazolium/formazan assay using 3-(4,5-dimethylthiazol-2-yl)-5-(3-carboxymethoxylphenyl)-2-(4-sulfophenyl)-2H-tetrazolium (MTS), and the like, can be used. For instance, when a significant decrease in neuronal intracellular ATP concentration or a significant increase in the level of neuronal mitochondrial damage is shown due to microglia stimulated by a test compound, the test compound can be determined to have neuronal death inhibitory activity.

Such screening methods of the present teachings screen for neuronal cell death inhibitors; however, they are preferred methods particularly suited for screening agents for the prevention and treatment of nervous system diseases, and can screen for agents for the prevention and treatment of the various nervous system diseases described above. In particular, agents for the prevention and treatment of nervous system diseases highly selective for neurotoxic microglia.

### EXAMPLES

Hereinafter, the present teachings will be described by giving examples; however, the present teachings is not limited to the following examples.

### Example 1

### Induction of Neuritic Beading Degeneration and Neuronal Cell Death Mediated by Cytokine-Stimulated Microglia Activation

In the present example, neuritic beading degeneration and neuronal cell death were observed in neurons when the neurons were administered with the microglia conditioned medium stimulated with various cytokines. The experimental methods were as follows.

### (1) Preparation of Microglia

Mouse primary microglia were isolated from primary mixed glial cell cultures (obtained from newborn C57BL/6J mice brains) by the ‘shaking off’ method on the 14th culture day or later (Suzumura, A. et al. MHC antigen expression on bulk isolated macrophage-microglia from newborn mouse brain: induction of 1a antigen expression by gamma-interferon. J. Neuroimmunol. 15, 263-278 (1987)).

### (2) Preparation of Neurons

In addition, mouse cerebral cortex primary neurons were prepared from the cerebral cortices of C57BL/6J mice at embryonic 17th day, and were plated on poly-ethyleneimine (PEI)-coated cover slips. Neurons were used at 10th to 13th culture day (Takeuchi et al. Neuritic beading induced by activated microglia is an early feature of neuronal dysfunction toward neuronal death by inhibition of mitochondrial respiration and axonal transport. J. Biol. Chem. 280, 10444-10454 (2005)).

### (3) Microglia Activation by Various Cytokines

Microglia were cultured with the culture medium (approximately 5×10⁴ cells/well, Neuron Medium (manufactured by Sumitomo Bakelite Co., LTD.) administered with 1 μg/ml of LPS or 100 ng/ml of cytokines (IL-1β, IL-6, IL-10, IFN-γ, or TNF-α), respectively. Microglia were incubated under 100% humidity and 5% CO₂ at 37° C. for 24 hours. Note that as a control, microglia were cultured similarly except that no cytokine was added.

### (4) Transmission of Stimulation to Neurons

### (a) Administration of Activated Microglia Conditioned Medium to Neurons (Indirect Stimulation Group)

Neurons in a 24-well plate (5×10⁴ cells/well) were incubated with 500 μl of conditioned medium of microglia activated as above. Neurons were administered similarly non-activated microglia conditioned medium to serve as a control for the indirect stimulation group. In addition, some neurons were added with 10 μM of MK801, which is an NMDA receptor antagonist. These neurons were cultured under 100% humidity and 5% CO₂ at 37° C.

### (b) Administration of Cytokines to Neurons (Direct Stimulation Group)

Neurons in a 24-well plate (5×10⁴ cells/well) were incubated with 500 μl of neuron culture medium containing 1 μg/ml of LPS or 100 ng/ml of cytokines (IL-1, IL-6, IL-10, IFN-γ, or TNF-α), respectively. In addition, neurons were similarly administered with only 500 μl of culture medium to serve as a control for the direct stimulation group. These were cultured under 100% humidity and 5% CO₂ at 37° C.

(5) Evaluation of the Number of Neurons with Neuritic Beading Degeneration and Dead Neurons

The various neurons obtained as above were cultured for 24 hours, then, neurons in each well were measured for both the number of neurons with neuritic beading degeneration and the number of dead neurons. To assess the number of neurons with neuritic beading degeneration, neurons were observed with a phase contrast microscopy. The ratio of neurons with neuritic beading degeneration was calculated as a percentage of total neurons. Note that neurons in duplicate wells were assessed blindly in three independent trials. In addition, the number of dead neurons was assessed by the dye-exclusion method with propidium iodide (PI). Neurons were incubated with the culture medium containing 2 mg/ml PI for 15 minutes at 37° C., then, they were observed with a conventional fluorescent microscope. The ratio of dead neurons was calculated as a percentage of PI-positive cells among total neurons. Moreover, the number of dead neurons was also evaluated with the terminal deoxynucleotidyl transferase-mediated UTP end labeling (TUNEL) staining.

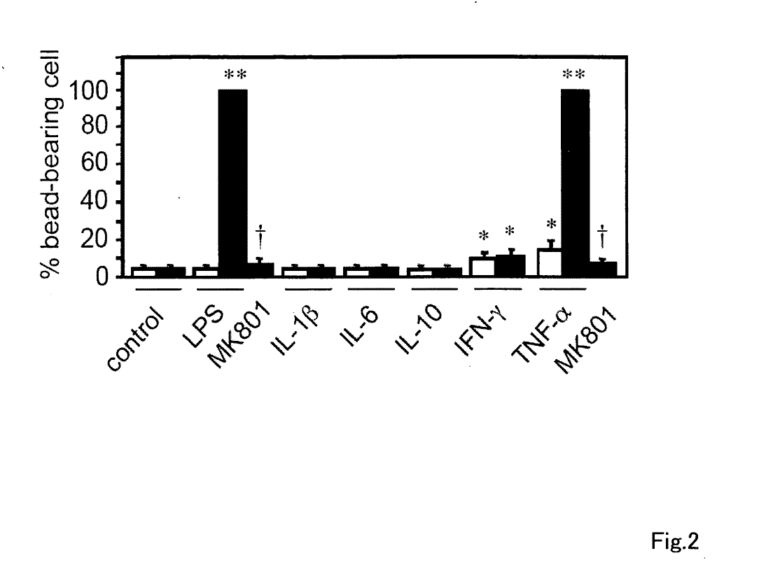

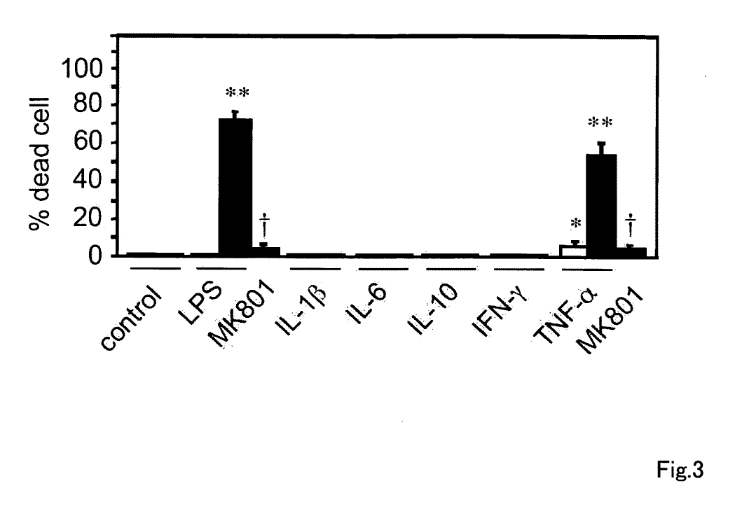

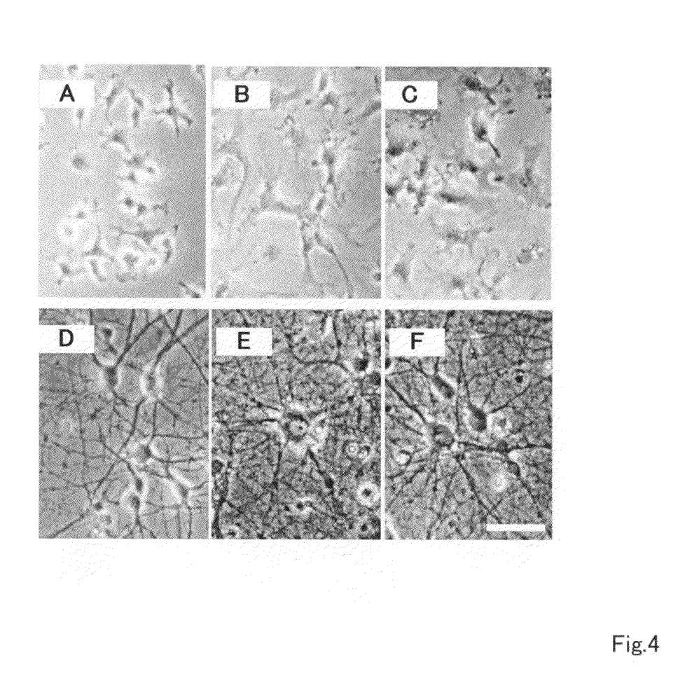

To assess the number of neurons with neuritic beading degeneration and dead neurons, neurons in duplicate wells administered with an identical culture medium were assessed blindly in three independent trials. Note that the ratio of dead neuron is calculated as a percentage of dead neurons among total neurons. The result of measurements of the number of neurons with neuritic beading degeneration is shown in FIG. 2, and the number of dead neurons is shown in FIG. 3. In addition, phase contrast microscopic images of microglia and neurons incubated with various stimuli are shown in FIG. 4.

### (6) Results

As shown in FIG. 2, neurons incubated with LPS- or TNF-α-treated microglia conditioned medium (indirect stimulation group) showed a significant decrease in neuritic beading degeneration (p<0.01 versus control), which ratio was approximately 100%. In addition, under the co-presence of MK801, which is an NMDA receptor antagonist, the degeneration was remarkably inhibited. In contrast, in the indirect stimulation groups of other cytokines and all direct stimulation groups, the positive rate was to a same extent to the control. Moreover, as shown in FIG. 3, similar results were obtained in the assessment of dead neurons (p<0.01 versus control).

As shown in FIG. 4, LPS- or TNF-α-treated microglia (FIGS. 4B and 4C) changed to a larger amoeboid morphology, exhibited a strong migrating activity, and was in an extremely active state, compared to non-stimulated microglia (FIG. 4A). In addition, numerous neuritic beads were observed in neurons incubated with LPS— and TNF-α-treated microglia conditioned medium (FIGS. 4E and 4F) compared to neurons incubated with non-stimulated microglia conditioned medium (FIG. 4 (D)). Note that TUNEL-positive cells were not observed, confirming that the cell death was not due to apoptosis.

From the above, it was revealed that neuritic beading degeneration and subsequent neuronal cell death occur, due to LPS or TNF-α among various cytokines, not directly but by indirect stimulation mediated by microglia activation. In addition, from the fact that MK801 inhibited such phenomenon, it was revealed that these phenomena were due to glutamate stimulation via the NMDA receptor.

### Example 2

### Increase in the Amount of Glutamate Released, Increase in Neuronal Intracellular ATP Concentration, and Increase in Mitochondrial Damage Mediated by Various Cytokines

In the present example, the amount of glutamate released from microglia stimulated with various cytokines, intracellular ATP concentration and mitochondrial damage in neurons incubated with conditioned medium were measured. The experimental methods were carried out similarly to Example 1 for the preparation of microglia, the preparation of neuron, the activation of microglia and transmission of stimulation to neuron (except that MK801 is not used). The evaluations were carried out by the following methods.

### (1) Measurement of Glutamate Concentration

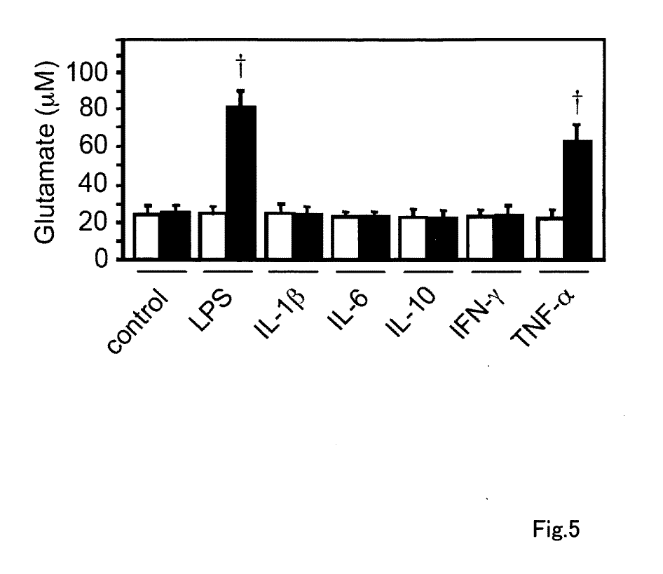

After incubation for 24 hours as above, the concentration of glutamate in the conditioned medium of each neuronal culture well was measured using Glutamate Assay Kit colorimetric assay (manufactured by Yamasa Corporation) according to the protocol thereof, measuring the absorption at 600 nm in a multiplate reader. Note that the assays were carried out in six independent trials. The results are shown in FIG. 5.

### (2) Measurement of Neuronal Intracellular ATP Concentration

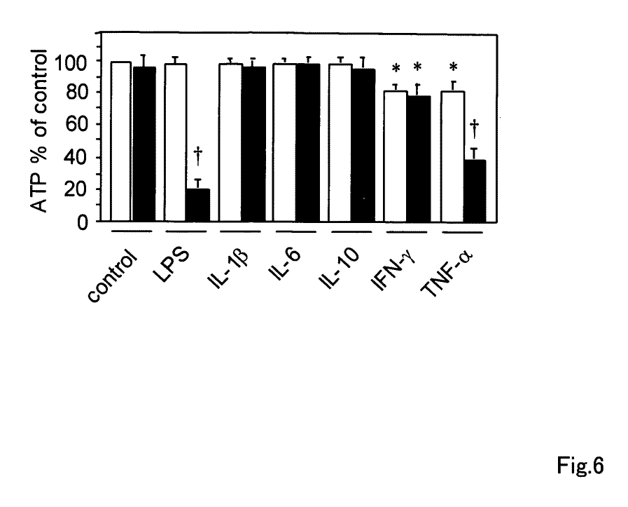

After incubation of the various neurons obtaine for 24 hours as above, intracellular ATP in each neuronal culture well was measured using AposSENSOR Cell Viability Assay Kit (manufactured by Bio Vision) according to the protocol thereof, by the chemiluminescent method. ATP concentration was calculated as a percentage of control. The results are shown in FIG. 6.

### (3) Measurement of Mitochondrial Damage

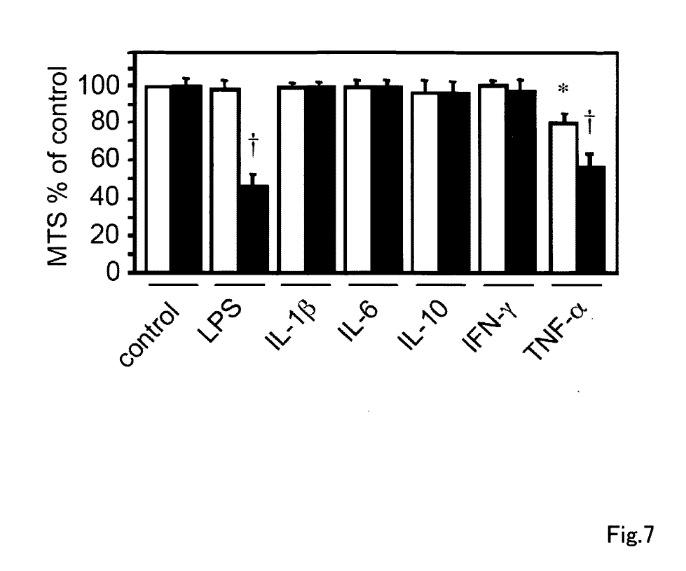

After incubation for 24 hours as above, the extent of neuronal mitochondria damage in each neuronal culture well was measured using CellTiter96 Aqueous One Solution assay (manufactured by Promega) according to the protocol, performing the MTS method, measuring the absorption at 490 nm in a multiplate reader. Note that the assays were carried out in six independent trials. The results are shown in FIG. 7.

### (4) Results

As shown in FIG. 5, glutamate was in significantly high concentration only in neuronal culture wells incubated with conditioned media from LPS- or TNF-α-treated microglia (p<0.01 with respect to neuron cultured in culture supernatant of LPS or TNF-α activated microglia). This is considered to be a reflection of the glutamate concentration contained in activated microglia conditioned medium. That is to say, it was considered that glutamate production and release in microglia activated by LPS or the like were accelerated, resulting in the glutamate concentration elevation in the microglia culture medium, and the glutamate concentration was reflected in the neuronal culture medium. In addition, as shown in FIG. 6, neuronal intracellular ATP concentration was significantly low only in neuronal culture wells incubated with conditioned media from LPS- or TNF-α-treated microglia (p<0.01 with respect to neuron cultured in culture supernatant of LPS or TNF-α activated microglia). Moreover, as shown in FIG. 7, the extent of mitochondrial damage was significantly mild only in neuronal culture wells incubated with conditioned media from LPS- or TNF-α-treated microglia (p<0.01 with respect to neuron cultured in culture supernatant of LPS or TNF-α activated microglia).

As described above, in the present example, it was revealed that LPS- or TNF-α-stimulated microglia increase glutamate released and induce decreases in neuronal intracellular ATP concentration and neuronal MTS level. In addition, from the results of Example 1 and Example 2, neuronal cell death or various signals related thereto are induced by the indirect stimulation via LPS- or TNF-α-stimulated microglia, i.e. due to the glutamate released by activated microglia.

### Example 3

### Inhibition of Glutamate Release by TNF-α-Neutralizing Antibody and TNF-α Receptor Type 1-Neutralizing Antibody

In the present example, neuritic beading degeneration and neuronal cell death were observed in neurons incubated with activated microglia conditioned medium in the presence of TNF-α-neutralizing antibody and TNF-α Receptor Type 1-neutralizing antibody. As the experimental methods, preparation of microglia and neurons was carried out similarly to Example 1, and microglia activation, transmission of stimulation to neurons and evaluation were as follows.

### (1) Activation of Microglia by LPS or TNF-α

LPS or TNF-α was added to microglia culture medium (approximately 5×10⁴ cells/well, Neuron Medium (manufactured by Sumitomo Bakelite)), so as to obtain 1 μg/ml for LPS and 1 ng/ml, 10 ng/ml and 100 ng/ml for TNF-α, and microglia were incubated under 100% humidity and 5% CO₂ at 37° C. for 24 hours.

### (2) Transmission of Stimulation to Neurons

Neurons in a 24-well plate (5×10⁴ cells/well) were incubated with 500% of activated microglia conditioned medium. In addition, neurons in a 24-well plate (5×10⁴ cells/well) were administered with 500 μl of activated microglia conditioned medium (100 μg/ml administration group only for TNF-α) in the presence of neutralizing antibody shown in the following table so as to obtain the final concentration listed in the table below. Note that non-activated microglia conditioned medium was similarly administered to neurons to serve as control. These neurons were cultured under 100% humidity and 5% CO₂ at 37° C.

TABLE 1

| | |
|---|---|
| TNF-α-neutralizing antibody | 0.1 mg/ml |
| TNF-α Receptor Type 1-neutralizing | 20 μg/ml |
| TNF-α Receptor Type 2-neutralizing | 20 μg/ml |

### (3) Evaluation

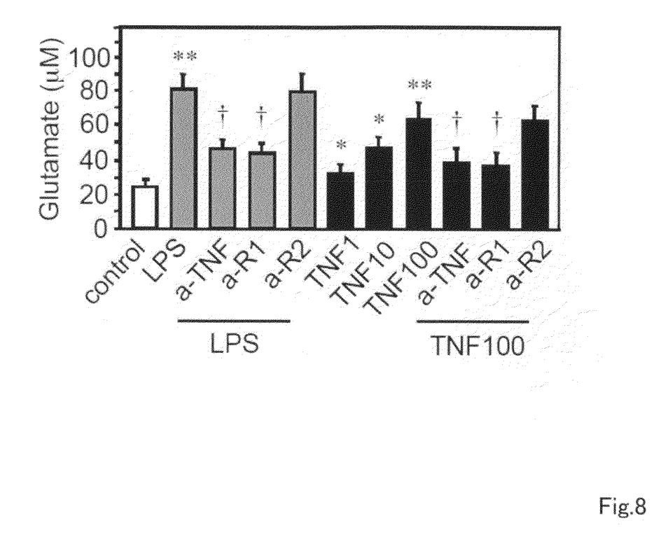

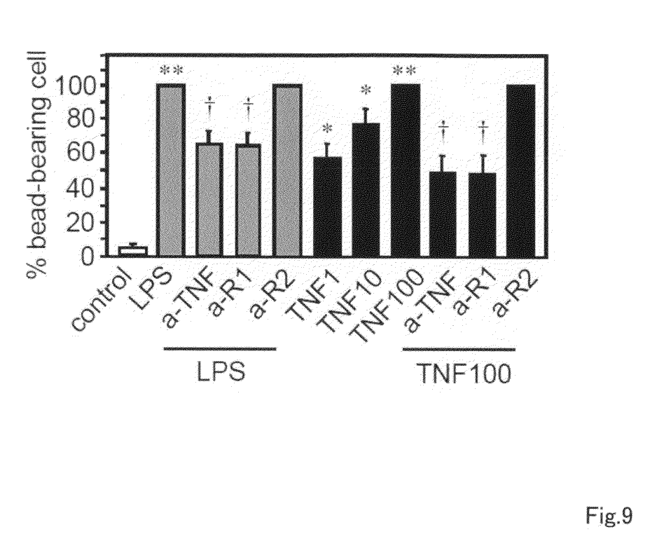

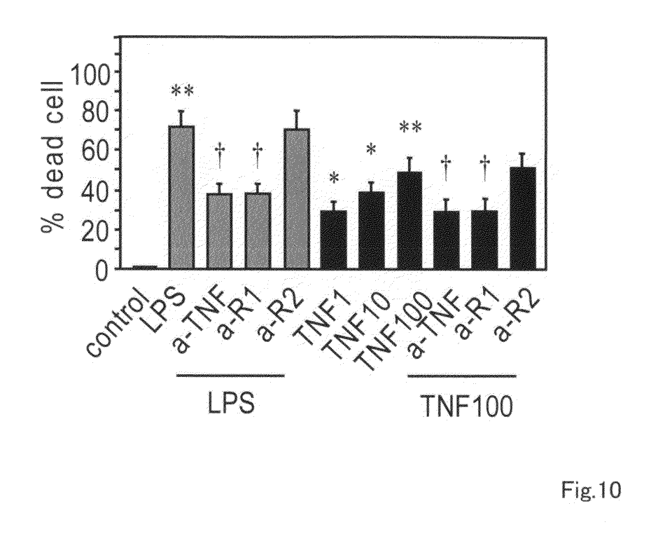

The various neurons prepared as above were cultured for 24 hours, then, glutamate concentration, the numbers of neurons with neuritic beading degeneration and dead neurons were measured for neurons in each neuronal culture well. Quantification of glutamate concentration was carried out similarly to Example 2, measurements of the numbers of neurons with neuritic beading degeneration and dead neurons were carried out similarly to Example 1. Result regarding glutamate concentration is shown in FIG. 8, result regarding neuritic beading degeneration is shown in FIG. 9, and result regarding dead neurons is shown in FIG. 10.

### (4) Results

As shown in FIG. 8, when TNF-α-neutralizing antibody or TNF-α Receptor Type 1-neutralizing antibody was present in activated microglia, glutamate concentration was significantly less than other neuronal culture media (p<0.05 versus neurons incubated with LPS or TNF-α-treated microglia conditioned medium). This was considered to reflect the glutamate concentration that was contained in the microglia conditioned medium. That is to say, these neutralizing antibodies inhibited microglia activation by TNF-α, as a result, microglial glutamate production was inhibited, decreasing the glutamate concentration in the microglia conditioned medium, and this glutamate concentration was reflected in the neuronal culture medium. As shown in FIGS. 9 and 10, similarly to the amount of glutamate, a significant inhibitory action was also observed regarding the numbers of neurons with neuritic beading degeneration and dead neurons (p<0.05 versus neurons incubated with LPS- or TNF-α-treated microglia conditioned medium).

From the above, it was revealed that glutamate release from activated microglia was inhibited by TNF-α-neutralizing antibody or TNF-α Receptor Type 1-neutralizing antibody, while at the same time, neuritic beading degeneration and cell death were also inhibited.

### Example 4

### Inhibition of TNF-α Induced Microglial Glutamate Production by Glutamine Elimination from Culture Medium, Glutaminase Inhibitor and Gap Junction Inhibitor

In the present example, glutamate release from microglia was measured and neuritic beading degeneration and cell death were observed when activated microglia and a variety of drugs were administered to neurons. As the experimental methods, preparation of microglia and neurons was carried out similarly to Example 1, and other processes were as follows.

(1) To activate microglia, a final concentration of 1 μg/ml LPS or 100 ng/ml TNF-α was administered to microglial culture medium (approximately 5×10⁴ cells/well, Neuron Medium (manufactured by Sumitomo Bakelite)), and microglia were incubated under 100% humidity and 5% CO₂ at 37° C. for 24 hours. Note that, as a control, microglia was incubated similarly except that no cytokine was added.

### (2) Transmission of Stimulation to Neurons

Neurons prepared in a 24-well plate (5×10⁴ cells/well) were incubated with 500 μl of microglia conditioned medium stimulated for 24 hours, along with various drugs shown in the following table (listed with final concentrations). In addition, neurons incubated with activated microglia conditioned medium but not containing glutamine in the culture medium (Gln-free) were also prepared. Moreover, neurons incubated with TNF-α-activated microglia conditioned medium alone and neurons incubated with non-activated microglia conditioned medium served respectively as TNF and control. These neurons were cultured under 100% humidity and 5% CO₂ at 37° C.

TABLE 2

| Symbol | Species | Compound Name | Concentration | |
|---|---|---|---|---|
| a-p38 | p38 MAPK inhibitor | SB203580 | 10 | μM |
| a-MEK | MEK inhibitor | PD98059 | 10 | μM |
| a-JNK | JNK inhibitor | | 10 | μM |
| a-IKK | IκB kinase inhibitor; | | 100 | μg/ml |
| THA | glutamate transporter | DL-threo-β- | 100 | μM |
| | inhibitor | hydroxyaspartic acid | | |
| CBX | gap junction inhibitor | carbenoxolone | 100 | μM |
| | | disodium(CBX) | | |
| DON | glutaminase inhibitor | 6-diazo-5-oxo-L- | 1 | mM |
| | | norleucine(DON) | | |

### (3) Evaluation

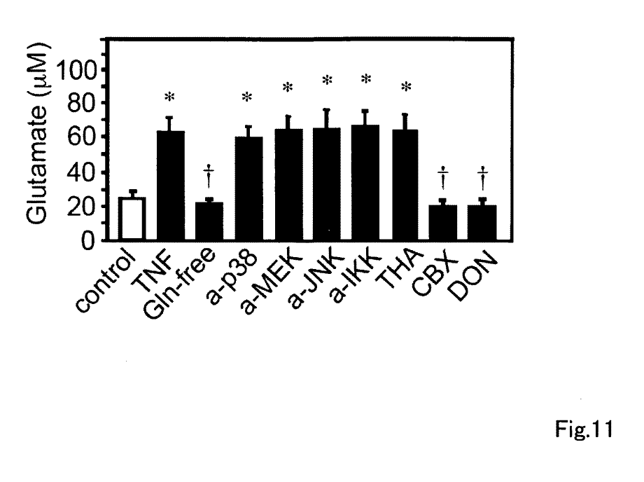

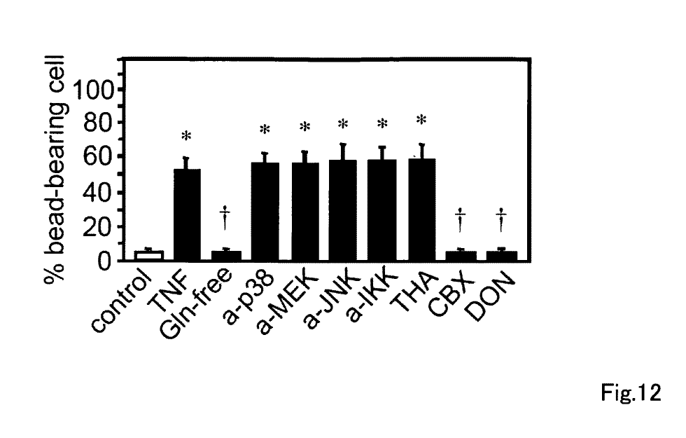

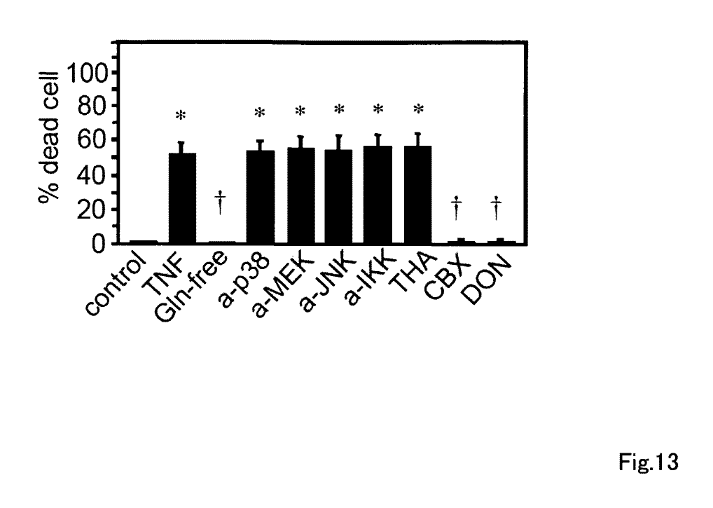

After 24-hour incubation, the glutamate concentration in the culture medium was measured, and the numbers of neurons with neuritic beading degeneration and dead neurons were also assessed. The methods described in Example 1 and Example 2 were used as the assessment. The result of glutamate concentration is shown in FIG. 11, the result of the number of neurons with neuritic beading degeneration is shown in FIG. 12, and the result of the number of dead neurons is shown in FIG. 13.

### (4) Results

As shown in FIG. 11, in the neurons incubated with activated microglia conditioned medium along with a gap junction inhibitor (CBX) and a glutaminase inhibitor (DON), and in the neurons incubated with glutamine-free microglia conditioned medium, extracellular glutamate concentrations were significantly (p<0.05) reduced to the control level compared to the neurons with no drug added (TNF). In regard to glutaminase inhibitor and gap junction inhibitor, it was considered that, in the presence thereof, microglial glutamate production and release were inhibited, decreasing the glutamate concentration, and this glutamate concentration was reflected in the neuronal culture medium. In addition, as shown in FIGS. 12 and 13, similarly to the amount of glutamate, significant (p<0.05) inhibitory action was also observed regarding the numbers of neurons with neuritic beading degeneration and dead neurons.

From the above, glutamine elimination from the culture medium, glutaminase inhibitor and gap junction inhibitor were shown to completely inhibit only the extra portion of microglial glutamate production induced by TNF-α, without perturbing the physiological basal level of intracellular glutamate production.

### Example 5

### Analysis of Gap Junction Expression

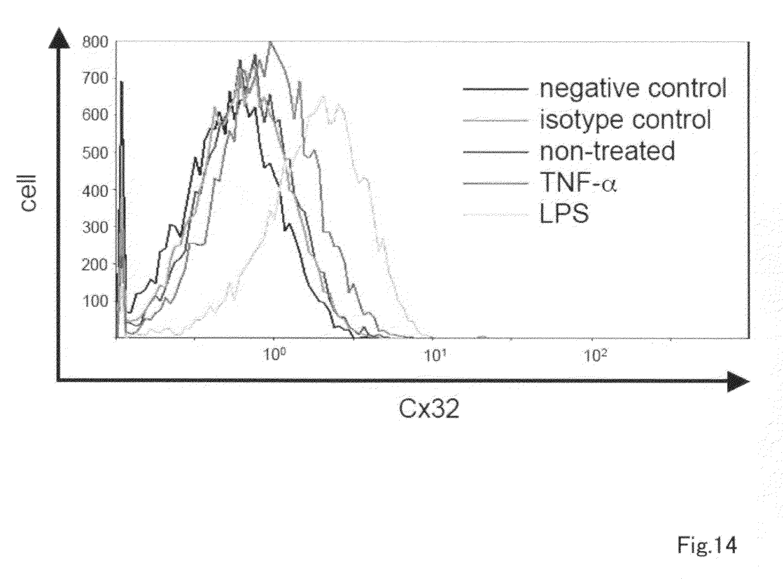

In the present example, an analysis of LPS- or TNF-α-stimulated microglial cell surface expression of connexin-32 (C×32), which is a major constitutive component of gap junction, was carried out with a flow cytometer. The preparation of microglia was carried out similarly to Example 1, and microglia activation was carried out similarly to Example 4. To Detect C×32 anti-mouse C×32 antibody (manufactured by Chemicon) was used. The result is shown in FIG. 14.

As shown in FIG. 14, expression of gap junction onto the cell surface of microglia was shown to be augmented by LPS or TNF-α.

### Example 6

In the present example, the effect of gap junction inhibitor and glutaminase inhibitor on neuronal cell death was evaluated using ischemia-induced delayed neuronal cell death model. Note that all protocols were approved by the Animal Experiment Committee of Nagoya University. Note that the animal model in the present example corresponds to a model of ischemic disorder, which is a nervous system disease.

According to reference by Imai et al. (Imai F, Sawada M, Suzuki H, Zlokovic B V, Kojima J, Kuno S, Nagatsu T, Nitatori T, Uchiyama Y, Kanno T., Exogenous microglia enter the brain and migrate into ischemic hippocampal lesions. et al. Neuroscience Letter. 272 (2): 127-130. 1999)□ adult male Mongolian gerbils, 10-12 weeks old and weighing approximately 70 g, were anesthetized with sevoflurane maintaining rectal temperature at 37° C. Global forebrain ischemia was produced transiently by occluding both common carotid arteries for 5 minutes using aneurysm clips.

Administration of the gap junction inhibitor carbenoxolone (CBX) was carried out in the following three groups. That is to say, the doses were 20 mg/kg body weight (CBX1), 2 mg/kg body weight (CBX1/10) and 0.2 mg/kg body weight (CBX1/100). Administration of the glutaminase inhibitor 6-diazo-5-oxo-L norleucine (DON) was carried out in the following three groups. That is to say, the doses were 1.6 mg/kg body weight (DON1), 0.16 mg/kg body weight (DON1/10) and 0.016 mg/kg body weight (DON1/100). CBX or DON was administered intraperitoneally every other day from the day of ischemia. Note that control animals were injected with the equal volume of phosphate-buffered saline (PBS).

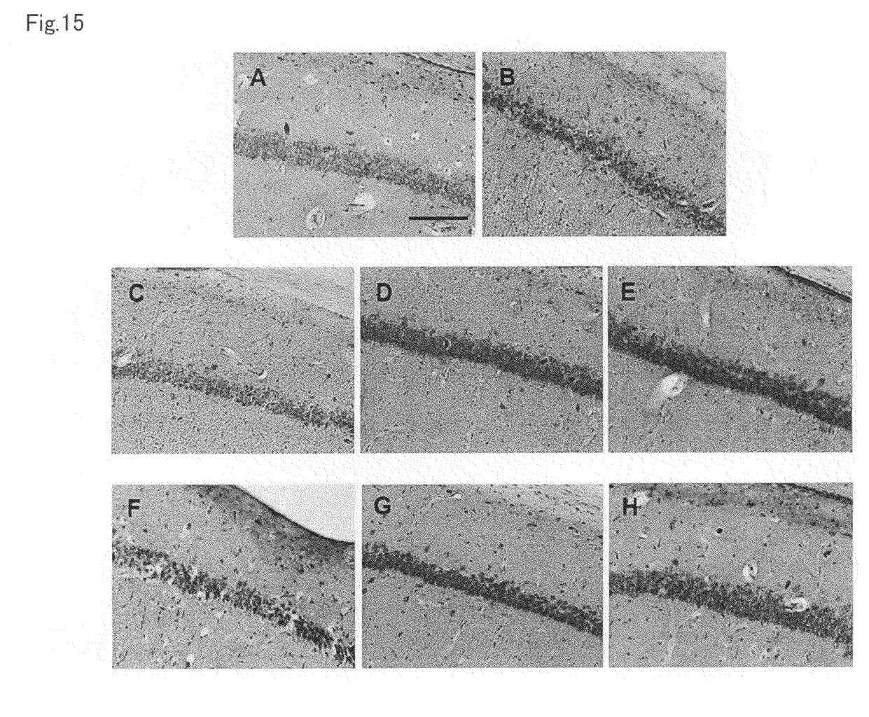

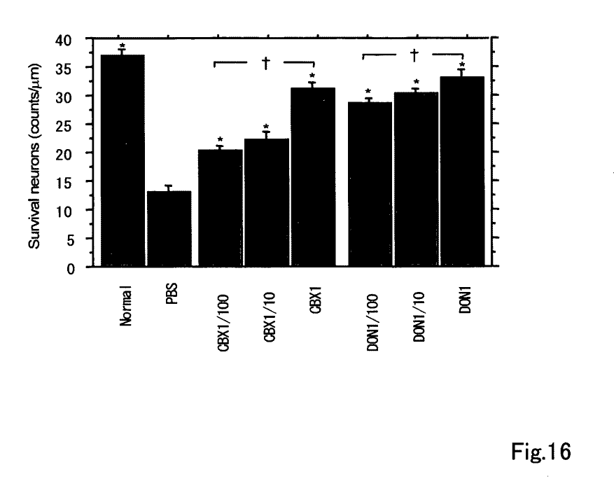

Seven days after ischemia, gerbils were anesthetized and transcardically perfused with 4% paraformaldehyde in PBS. The brains were removed, embedded in O.C.T. compound (manufactured by Sakura Finetech) and then frozen in liquid nitrogen. Frozen sections were prepared with a cryostat (8 μm thick), were mounted onto a slide glass and were stained with haematoxylin and eosin. Microscopic image in each administered group is shown in FIG. 15. To assess the effect of drug treatment on delayed neuronal death, the number of surviving neurons per 100 μm in the hippocampal CA1 region was counted under a microscope. The result for each administered group is shown in FIG. 16.

As shown in FIG. 15, administration of gap junction inhibitor or glutaminase inhibitor clearly inhibited the delayed neuronal cell death in the ischemia-induced delayed neuronal cell death model. In addition, as shown in FIG. 16, administration of CBX or DON significantly protected the number of surviving neurons per unit area of gerbil hippocampal CA1 region (p<0.001 versus control). Moreover, both CBX and DON decreased neuronal death in a dose-dependent manner.

From the above, it was revealed that gap junction inhibitor and glutaminase inhibitor both are able to inhibit neuronal cell death, especially neuronal cell death in the central nervous system. In addition, the neuronal death inhibitor of the present invention was shown to be effective for the prevention and treatment of ischemic disorders such as brain hemorrhage and cerebral infarction, and sequelae of ischemic disorder such as cerebrovascular dementia.

### Example 7

In the present example, using myelin oligodendrocyte glycoprotein (MOG)-induced experimental autoimmune encephalomyelitis (EAE) model, the effects of gap junction inhibitor and glutaminase inhibitor on EAE clinical symptoms were evaluated. Note that all protocols were approved by the Animal Experiment Committee of Nagoya University. Note that the animal model in the present example corresponds to a model of neuroinflammatory disease, which is a nervous system disease.

C57BL/6J mice (purchased from Japan SLC) were used as experimental animals. In addition, MOG₃₅₋₅₅ peptide (manufactured by Operon), incomplete Freund's adjuvant (manufactured by Sigma), heat-killed bacteria Mycobacterium tuberculosis H37Ra (manufactured by Difco), pertussis toxin (manufactured by List), gap junction inhibitor carbenoxolone (CBX) (manufactured by Sigma) and glutaminase inhibitor 6-diazo-5-oxo-norleucine (DON) (manufactured by Sigma) were used as reagents.

MOG-induced EAE was prepared as the reference by Kato et al. (Kato, H., Ito, A., Kawanokuchi, J., Jin, S., Mizuno, T., Ojika, K., Ueda, R., Suzumura A., Pituitary adenylate cyclase-activating polypeptide (PACAP) ameliorates experimental autoimmune encephalomyelitis by suppressing the functions of antigen presenting cells. et al. Multiple Sclerosis. 10, 651-659. (2004)). 200 μg of MOG₃₅₋₅₅ peptide was dissolved in 100 μl of saline. In addition, 300 μg of heat-killed bacteria Mycobacterium tuberculosis H37Ra were suspended in 100 μl of incomplete Freund's adjuvant. Then, both were mixed and emulsified. C57BL/6J mice aged 6-8 weeks were immunized subcutaneously at the base of the tail with 200 μl of this emulsion. Next, mice were injected with 200 ng of pertussis toxin intraperitoneally on the immunization day and two days after immunization.

Administration of the gap junction inhibitor carbenoxolone (CBX) was carried out in the following three groups. That is to say, the doses were 20 mg/kg body weight (CBX1), 2 mg/kg body weight (CBX1/10) and 0.2 mg/kg body weight (CBX1/100). Administration of the glutaminase inhibitor 6-diazo-5-oxo-L norleucine (DON) was carried out in the following three groups. That is to say, the doses were 1.6 mg/kg body weight (DON1), 0.16 mg/kg body weight (DON1/10) and 0.016 mg/kg body weight (DON1/100). CBX or DON was administered intraperitoneally every other day from the day of immunization. Note that control animals were injected with the equal volume of phosphate-buffered saline (PBS).

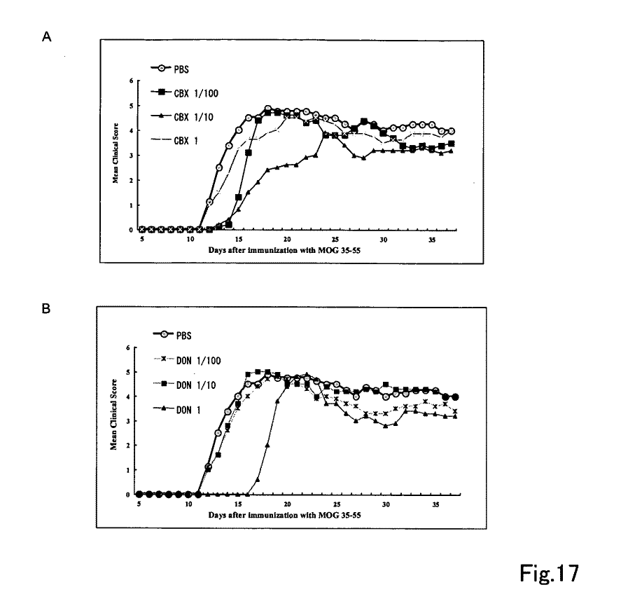

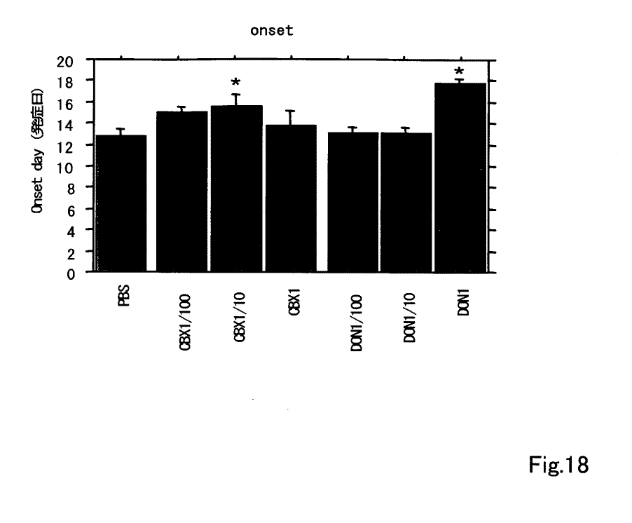

Mice were evaluated daily for clinical signs of EAE using the following scale, which is internationally accepted. EAE clinical course of each administered group is shown in FIG. 17, and the results of the EAE onset day, the number of severe sick days and the peak clinical score are shown in FIG. 18 to FIG. 20.

EAE clinical score
0: normal
1: limp tail or mild hind limb weakness
2: mild hind limb weakness or mild ataxia
3: moderate to severe hind limb weakness
4: severe hind limb weakness, mild forelimb weakness or mild ataxia
5: paraplegia accompanied by mild forelimb weakness
6: paraplegia accompanied by severe forelimb weakness or severe ataxia, or moribundity

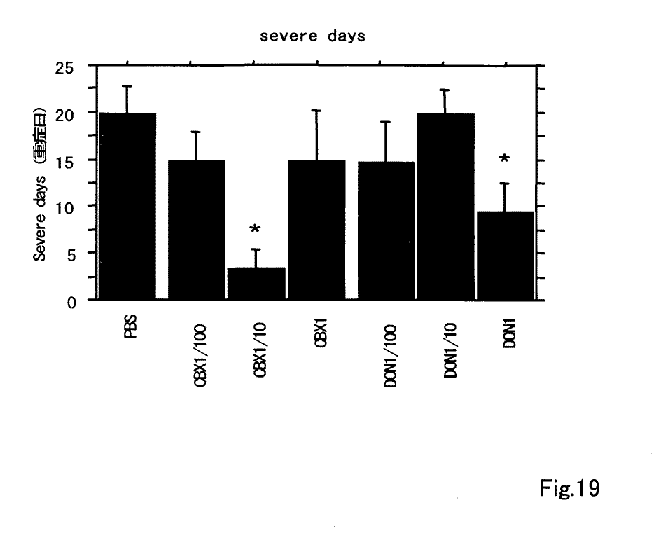

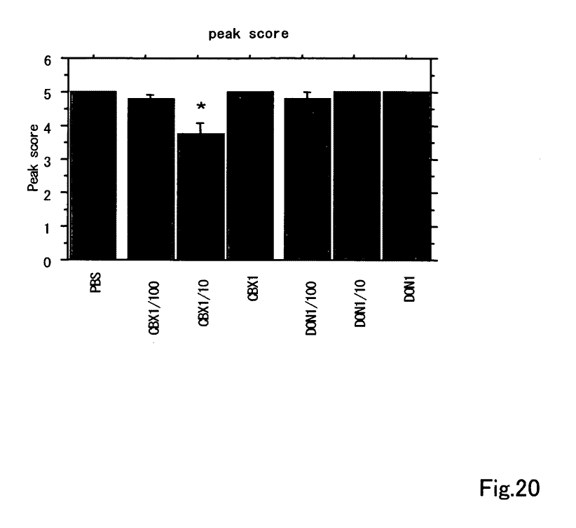

As shown in FIG. 17, administration of gap junction inhibitor or glutaminase inhibitor inhibited EAE clinical symptoms. In addition, as shown in FIG. 18, according to the clinical course shown in FIG. 17, the EAE onset day (when EAE clinical score becomes 1 or greater) was significantly delayed (p<0.05) in CBX1/10-administrated group and DON1-administrated group. In addition, as shown in FIG. 19, the number of severe sick days (EAE clinical score is four or greater) was significantly reduced (p<0.05) in CBX1/10-administrated group and DON1-administrated group. Furthermore, as shown in FIG. 20, the peak clinical score was significantly decreased in CBX1/10-administrated groups. From the above results, it was revealed that gap junction inhibitor and glutaminase inhibitor both are able to inhibit neuronal cell death, especially neuronal cell death in the central nervous system.

1-25. (canceled)

26. A screening method for an inhibitor that inhibits cell death of neuron, the method comprising:
administrating a test compound to activated microglia; and
evaluating effects of one or more inhibitory activities of inhibiting glutamate production and/or release from activated microglia.

27. The method according to claim 26, wherein said one or more inhibitory activities are selected from the following (1) and (2):
(1) inhibitory activity of glutaminase of activated microglia; and
(2) inhibitory activity of gap junction of activated microglia.

28. The screening method according to claim 26, wherein each of the inhibitory activities is within a range that maintains the amount of glutamate produced to level with the amount of glutamate produced when microglia is not activated.

29. The screening method according to claim 28, wherein LPS- or TNF-α-stimulated microglia are used as activated microglia.

30. A screening method for a prophylactic and therapeutic agent of an neuroinflammatory disease, the method comprising:
administrating a test compound to activated microglia; and
evaluating effects of one or more inhibitory activities of inhibiting glutamate production and/or release from activated microglia.

31. The method according to claim 30, wherein the one or more inhibitory activities are selected from the following (1) and (2):
(1) inhibitory activity of glutaminase of activated microglia; and
(2) inhibitory activity of gap junction of activated microglia.

32. The screening method according to claim 30, wherein each of the inhibitory activities are within a range that maintains the amount of glutamate produced to the level with the amount of glutamate produced when microglia is not activated.

33. The screening method according to claim 30, wherein LPS- or TNF-α-stimulated microglia are used as activated microglia.

34. The screening method according to claim 30, wherein neuroinflammatory disease is selected from acute disseminated encephalomyelitis, sequelae of encephalitis, bacterial meningitis, tuberculous meningitis, fungal meningitis, viral meningitis, post-vaccinal meningitis, and AIDS encephalopathy.

35. The screening method according to claim 34, wherein neuroinflammatory disease is multiple sclerosis.

36. A method for preventing or treating an neuroinflammatory disease, the method comprising:
administrating an effective dose of one or more active ingredients selected from glutaminase inhibitors and gap junction inhibitors.

37. The method according to claim 36, wherein the active ingredients are administrated in the effective dose for inhibiting generation and/or release of glutamate in the activated microglia.

38. The method according to claim 37, wherein the one or more active ingredients are glutaminase inhibitors.

39. The method according to claim 37, wherein the one or more active ingredients are gap junction inhibitors.

40. The method according to claim 36, wherein said neuroinflammatory disease is selected from acute disseminated encephalomyelitis, sequelae of encephalitis, bacterial meningitis, tuberculous meningitis, fungal meningitis, viral meningitis, post-vaccinal meningitis, multiple sclerosisand AIDS encephalopathy.

41. The method according to claim 40, wherein said neuroinflammatory disease is multiple sclerosis.
